(19) United States
(12) Patent Application Publication
(10) Pub. No.: US 2026/0102379 A1
(43) Pub. Date: Apr. 16, 2026

(54) Inhibitor of PFKFB2 Kinase Activity and Cellular Glycolysis

(71) Applicants: Oklahoma Medical Research Foundation, Oklahoma City, OK (US); Atomwise Inc., San Francisco, CA (US)

(72) Inventors: Kenneth Humphries, Oklahoma City, OK (US); Craig Eyster, Guthrie, OK (US); Satoshi Matsuzaki, Oklahoma City, OK (US); Mostafa Ahmed, San Francisco, CA (US)

(21) Appl. No.: 19/356,875

(22) Filed: Oct. 13, 2025

Related U.S. Application Data
(60) Provisional application No. 63/706,835, filed on Oct. 14, 2024.

(51) Int. Cl. A61K 31/433; C07D 285/135

(52) U.S. Cl. CPC A61K 31/433; C07D 285/135

(57) ABSTRACT

Provided herein are compositions and method of treating a disease or condition with increased 6-phosphofructo-2-kinase/fructose-2,6-biphosphatase 2 (PFKFB2) activity comprising: administering to a subject in need of a treatment for the heart disease or cancer an effective amount of a selective inhibitor of 6-phosphofructo-2-kinase/fructose-2,6-biphosphatase 2 (PFKFB2) comprising: contacting PFKFB2 with a molecule of formula:

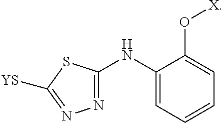

### CROSS-REFERENCE TO RELATED APPLICATIONS

This application claims priority to U.S. Provisional Application Ser. No. 63/706,835 filed Oct. 14, 2024, the entire contents of which are incorporated herein by reference.

### STATEMENT OF FEDERALLY FUNDED RESEARCH

This invention was made with government support under R01HL160955 awarded by the National Institutes of Health. The government has certain rights in the invention.

### TECHNICAL FIELD

The present invention relates in general to the field of inhibitors of kinase activity, and more particularly, to inhibitors of PFKFB2 kinase activity and cellular glycolysis.

### INCORPORATION-BY-REFERENCE OF MATERIALS FILED ON COMPACT DISC

Not applicable.

### BACKGROUND

Without limiting the scope of the invention, its background is described in connection with the 6-phosphofructo-2-kinase/fructose-2,6-biphosphatase (PFKFB) family of proteins.

The 6-phosphofructo-2-kinase/fructose-2,6-biphosphatase (PFKFB) family of proteins are bifunctional enzymes that catalyze the formation or degradation of fructose-2,6-bisphosphate (F-2,6-BP). F-2,6-BP is an allosteric activator of phosphofructokinase-1 (PFK-1), a committed and rate limiting step of glycolysis. Therefore, depending upon whether F-2,6-BP is being produced or degraded, the dual activities of PFKFB enzymes play a central role in increasing or decreasing glycolysis.

There are four PFKFB isoforms. In the heart, PFKFB2 is the primary isoform and its kinase activity is enhanced by protein phosphorylation by either Akt, AMPK, or PKA. The inventors have shown that cardiac PFKFB2 is decreased in diabetic mouse models and that its content is regulated by insulin signaling. The constitutive decrease of cardiac PFKFB2 likely contributes to diabetes-induced dysregulation of normal metabolic flexibility cues. Tissue-specific splice variants of PFKFB1 are expressed either in skeletal muscle or liver where they facilitate fine-tuned regulation of glycolysis and/or gluconeogenesis. In contrast to PFKFB2, protein phosphorylation of PFKFB1 by PKA increases phosphatase activity. PFKFB3 is an inducible member of the PFKFB family and is upregulated in numerous cancers, likely contributing to dysregulation of glucose metabolism and contributing to aerobic glycolysis. Lastly, PFKFB4 expression is normally confined to testes but its upregulation is implicated in cancers [1].

The central role of PFKFB enzymes in maintaining metabolic flexibility and regulating glucose metabolism make them potential targets for therapeutic intervention. However, progress on this front remains limited in part because of the difficulty in measuring PFKFB enzyme activity, the instability of the kinase product F-2,6-BP, and the lack of available commercial F-2,6-BP standards.

A need remains for a PFKFB2 inhibitor that has a reduced, or no effect, on other members of the 6-phosphofructo-2-kinase/fructose-2,6-biphosphatase family.

### SUMMARY

As embodied and broadly described herein, an aspect of the present disclosure relates to a method of inhibiting 6-phosphofructo-2-kinase/fructose-2,6-biphosphatase 2 (PFKFB2) comprising: contacting PFKFB2 with a molecule of formula:

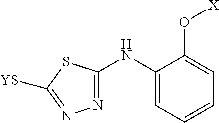

wherein X is CH₃, S, a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, a cyano group,
wherein Y is H, —SH, O, OH, O₂—R, S, a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, or a cyano group;
wherein R is a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, or a cyano group. In one aspect, the molecule has a reduced inhibitory effect against 6-phosphofructo-2-kinase/fructose-2,6-biphosphatase 3 (PFKFB3). In another aspect, the molecule molecule is

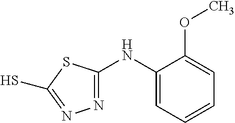

5-[(2-methoxyphenyl)amino]-1,3,4-thiadiazole-2-thiol, salts, and derivatives thereof. In another aspect, the molecule is formulated for intravenous, oral, enteral, parenteral, topical, intramuscular, subcutaneous, intradermal, transdermal, rectal, vaginal, mucosal, buccal, intraperitoneal, intranasal, sublingual, intranasal, intrapulmonary administration. In another aspect, the molecule further comprises one or more pharmaceutical acceptable carriers, excipients, buffers, binders, polymers, coatings, fillers, flavors, glidants, coatings, disintegrants, dyes, preservatives, sorbents, sweeteners, or salts. In another aspect, a dose for administration of the molecule is 0.01, 0.1, 1, 10, 20, 25, 30, 40, 50, 60, 70, 75, 80, 90, 100, 150, 150, 300, 333, 400, 500, 600, 700, 750, 800, 900, 1,000, 2,500, 5,000, 7,500, or 10,000 mg per dose. In another aspect, a dose for administration of the molecule is 0.01-0.1, 0.1-0.25, 0.1-0.4, 0.35-0.5, 0.5-1, 1-2, 1-3, 1-4, 1-5, 1-2.5, 2.5-3.5, 4-6, 5-8, 6-9, 7-10 grams per dose. In another aspect, the molecule is formulated for delivered orally via a mini-tablet, capsule, tablet, effervescent, dual release, mixed release, sachet, powder, or liquid. In another aspect, the molecule is formulated for daily doses of about 0.01, 0.1, 1, 2, 3, 4, 5, 10, 20, 25, 30, 40, 50, 60, 70, 75, 80, 90, 100 mg/Kg of body weight.

As embodied and broadly described herein, an aspect of the present disclosure relates to a method of treating a disease or condition with increased 6-phosphofructo-2-kinase/fructose-2,6-biphosphatase 2 (PFKFB2) activity comprising: administering to a subject in need for the disease or condition an effective amount of a 6-phosphofructo-2-kinase/fructose-2,6-biphosphatase 2 (PFKFB2) inhibitor comprising of formula:

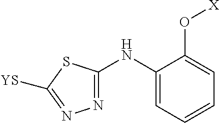

wherein X is CH₃, S, a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, a cyano group,
wherein Y is H, —SH, O, OH, O₂—R, S, a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, or a cyano group;
wherein R is a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, or a cyano group. In one aspect, the PFKFB2 inhibitor has a reduced activity against 6-phosphofructo-2-kinase/fructose-2,6-biphosphatase 3 (PFKFB3). In another aspect, the PFKFB2 inhibitor is:

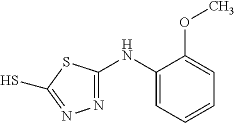

5-[(2-methoxyphenyl)amino]-1,3,4-thiadiazole-2-thiol, salts, and derivatives thereof. In another aspect, the PFKFB2 inhibitor is formulated for intravenous, oral, enteral, parenteral, topical, intramuscular, subcutaneous, intradermal, transdermal, rectal, vaginal, mucosal, buccal, intraperitoneal, intranasal, sublingual, intranasal, intrapulmonary administration. In another aspect, the PFKFB2 inhibitor further comprises one or more pharmaceutical acceptable carriers, excipients, buffers, binders, polymers, coatings, fillers, flavors, glidants, coatings, disintegrants, dyes, preservatives, sorbents, sweeteners, or salts. In another aspect, a dose of the PFKFB2 inhibitor for administration is 0.01, 0.1, 1, 10, 20, 25, 30, 40, 50, 60, 70, 75, 80, 90, 100, 150, 150, 300, 333, 400, 500, 600, 700, 750, 800, 900, 1,000, 2,500, 5,000, 7,500, or 10,000 mg per dose. In another aspect, a dose of the PFKFB2 inhibitor for administration is 0.01-0.1, 0.1-0.25, 0.1-0.4, 0.35-0.5, 0.5-1, 1-2, 1-3, 1-4, 1-5, 1-2.5, 2.5-3.5, 4-6, 5-8, 6-9, 7-10 grams per dose. In another aspect, the PFKFB2 inhibitor is delivered orally via a mini-tablet, capsule, tablet, effervescent, dual release, mixed release, sachet, powder, or liquid. In another aspect, the PFKFB2 inhibitor is administered in daily doses of about 0.01, 0.1, 1, 2, 3, 4, 5, 10, 20, 25, 30, 40, 50, 60, 70, 75, 80, 90, 100 mg/Kg of body weight. In another aspect, the PFKFB2 inhibitor is administered two or three times daily. In another aspect, the disease is a cancer selected from at least one of: glioblastoma multiforme, skin cancer, connective tissue cancer, fat cancer, breast cancer, lung cancer, stomach cancer, pancreatic cancer, ovarian cancer and genital cancer, cervix Cancer, uterine cancer, anogenital cancer, kidney cancer, bladder cancer, liver cancer, colorectal cancer or colon cancer and digestive (GI) duct cancer, prostate cancer and genital cancer, central nervous system (CNS) cancer, retinal cancer, blood Cancer, lymphocyte cancer, and head and neck cancer. In another aspect, the disease is myocardial ischemia, congestive heart failure, heart failure with preserved ejection fraction, or cardiac tissue regeneration. In another aspect, the PFKFB2 inhibitor decreased fructose-1,6-bisphosphate and increased fructose-6-phosphate levels in cells.

As embodied and broadly described herein, an aspect of the present disclosure relates to a molecule of formula:

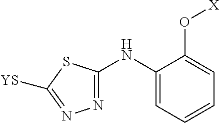

wherein X is CH₃, S, a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, a cyano group,
wherein Y is H, —SH, O, OH, O₂—R, S, a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, or a cyano group;
wherein R is a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, or a cyano group. In one aspect, the formula is not:

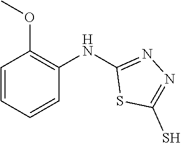

### BRIEF DESCRIPTION OF THE DRAWINGS

For a more complete understanding of the features and advantages of the present disclosure, reference is now made to the detailed description of the disclosure along with the accompanying figures and in which:

FIGS. 1A to 1D show that recombinant PFK2 is phosphorylated in vitro. (FIG. 1A) Western blot analysis of cleared bacterial lysates of GST-PFK2 and mutants. Top: Total protein staining of cleared bacterial lysates over-expressing GST-PFK2 and mutants. Bottom: Western blots against GST and phosphorylated-PFK2 for GST-PFK2 and mutants. (FIG. 1B) Western blot analysis of GST-PFK2 expression, purification, and in vitro phosphorylation. (FIG. 1C) Western blot analysis of GST-PFK2 and mutant proteins treated with PKA after purification. (FIG. 1D) Western blot against phosphorylated PFK2 of bacterial samples treated/not treated with λ-phosphatase for indicated times before lysis. Quantitative analysis of bacterial samples treated with λ-phosphatase. Data are shown as mean±SD.

FIGS. 2A to 2E show the development of a PFKFB kinase activity assay and screening of potential modulators. (FIG. 2A) Schematic of the coupled enzyme activity assay protocol. (FIG. 2B) Validation of the coupled assay comparing wild type (WT) and kinase-null K174G (K) PFKFB2 mutant. (FIG. 2C) Plate-reader screening of candidate compounds provided by Atomwise. (FIG. 2D) IC50 determination of candidate inhibitor B2 (structure on inset) for PFKFB2 (top) and PFKFB3 (lower) kinase activities. (FIG. 2E) IC50 determination of known PFKFB3 kinase inhibitor PFK158 (structure on inset) for PFKFB2 (top) and PFKFB3 (lower) kinase activities.

FIGS. 3A and 3B show that B2 inhibits glycolysis and glycolytic capacity. A-498 cells were treated with 5 μM B2 for 60 minutes. The rate of glycolysis (FIG. 3A) was measured as ECAR upon addition of glucose and glycolytic capacity (FIG. 3B) as the rate of ECAR after the subsequent addition of oligomycin. Each point represents an individual well from a Seahorse XFe24 plate, with the experiment performed on 2 separate plates. Data are shown as mean±SD. Not significant (ns): P>0.05, *P≤0.05, **P≤0.01, ****P≤0.0001 by one-way ANOVA with multiple comparison of the mean of each test group to the mean of the DMSO control.

FIGS. 4A and 4B show metabolic changes mediated by inhibited PFKFB2 activity in cells. (FIG. 4A) A-498 cells were treated with B2 or PFK158 for 60 min and then metabolites extracted for LC/MS analysis. Early glycolytic intermediates and the ratio of F-6-P to F-1,6-BP (lower right) are shown. Created with Biorender. (FIG. 4B) PCA analysis of control, B2 and PK158 treatment groups. Data are shown as mean±SD. Not significant (ns): **P≤0.01, ****P≤0.0001 by one-way ANOVA with multiple comparison of the mean of each test group to the mean of the vehicle control.

FIG. 5 shows a heatmap of targeted metabolomic data. Each column is a separate biological replicate (n=5). The box indicates the clustering of decreased downstream glycolytic intermediates in B2-treated cells. The ratio of fructose-6-phosphate (F-6-P) to fructose-1,6-bisphosphate (F-1,6-BP) is included in the heatmap. Other abbreviations: HGA, DL-hydroxyglutaric acid; PEP, phosphoenolpyruvic acid; 3-PGA, 3-phosphoglyceric acid; G3P; glyceraldehyde 3-phosphate; Data (n=5) are shown as mean±SD. Not significant (ns): **P≤0.01, ****P≤0.0001 by one-way ANOVA with multiple comparison of the mean of each test group to the mean of the vehicle control. PCA and heatmap were generated with MetaboAnalyst 6.0; created in BioRender.

### DETAILED DESCRIPTION

While the making and using of various aspects of the present disclosure are discussed in detail below, it should be appreciated that the present disclosure provides many applicable inventive concepts that can be embodied in a wide variety of specific contexts. The specific aspects discussed herein are merely illustrative of specific ways to make and use the disclosure and do not delimit the scope of the disclosure.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration. The terminology herein is used to describe specific aspects of the disclosure, but their usage does not delimit the disclosure, except as outlined in the claims.

The 6-phosphofructo-2-kinase/fructose-2,6-biphosphatase (PFKFB) family of proteins (PFKFB 1-4) are bifunctional enzymes that catalyze the formation or degradation of fructose-2,6-bisphosphate (F-2,6-BP). They are of clinical relevance because of their roles in regulating glycolysis in insulin sensitive tissues and their upregulation in numerous cancer types. The present disclosure used recombinant PFKFB2 to develop a robust enzymatic assay to test a library of small molecules selected by the Atomwise AtomNet AI platform. This resulted in the identification of a new inhibitor, termed here B2, that inhibits PFKFB2 (IC50 3.29 μM) and PFKFB3 (IC50 11.89 μM). The inventors tested the effects of B2 on A-498 cells, which express both PFKFB2 and PFKFB3. Seahorse XFe analysis of the extracellular acidification rates revealed B2 inhibited cellular glycolysis and glycolytic capacity. Under the same experimental conditions, LC/MS analysis revealed B2 decreased fructose-1,6-bisphosphate but increased fructose-6-phosphate levels, which is consistent with an inhibitory effect on PFK-1 activity. Interestingly, the metabolic profile of A-498 cells treated under identical conditions with known PFKFB3 inhibitor, PK158, was distinct from that induced by B2. Thus, it is demonstrated herein that the molecule is a new PFKFB kinase activity inhibitor. Furthermore, this is the first instance of a drug that was specifically developed with PFKFB2 as a target. Other inhibitors in the literature have focused on specifically targeting PFKFB3.

The PFKFB2 and PFKFB3 inhibitor of the present disclosure has the formula:

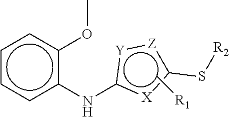

X is NH, CH2, S, a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, a cyano group;
Y is NH, CH2, CH—SH, C═O, CH—OH, S a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, a cyano group;
Z is NH, CH2, CH—SH, C═O, CH—OH, a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, a cyano group;
R1 is an optionally substituted ring selected from phenyl, a 4-, 5-, 6-, 7- or 8-membered saturated or partially unsaturated monocyclic carbocyclic ring, a 4-, 5-, or 6, 7- or 8-membered saturated or partially unsaturated monocyclic heterocyclic ring having 1-2 heteroatoms independently selected from nitrogen, oxygen, or sulfur, a 5-8 membered monocyclic heteroaromatic ring having 1, 2, 3, or 4 heteroatoms independently selected from nitrogen, oxygen, or sulfur, a 8-10 membered bicyclic aromatic ring, or a 8-10 membered bicyclic heteroaromatic ring having 1-5 heteroatoms independently selected from nitrogen, oxygen, or sulfur;
R2 is H, —S(O)₂NR3, —S(O)₂R3, —C(O)NR3, —C(O)R3, wherein R3 is an optionally substituted —C₁₋₁₂ aliphatic, a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, an optionally substituted ring selected from phenyl, a 4-, 5-, 6-, 7- or 8-membered saturated or partially unsaturated monocyclic carbocyclic ring, a 4-, 5-, or 6, 7- or 8-membered saturated or partially unsaturated monocyclic heterocyclic ring having 1-2 heteroatoms independently selected from nitrogen, oxygen, or sulfur, a 5-8 membered monocyclic heteroaromatic ring having 1, 2, 3, or 4 heteroatoms independently selected from nitrogen, oxygen, or sulfur, a 8-10 membered bicyclic aromatic ring, or a 8-10 membered bicyclic heteroaromatic ring having 1-5 heteroatoms independently selected from nitrogen, oxygen, or sulfur.

In one aspect, the molecule has reduced activity against 6-phosphofructo-2-kinase/fructose-2,6-biphosphatase 3 (PFKFB3) activity. In one specific example, the molecule is

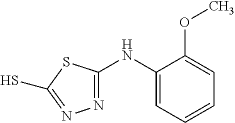

5-[(2-methoxyphenyl)amino]-1,3,4-thiadiazole-2-thiol, salts, and derivatives thereof; or

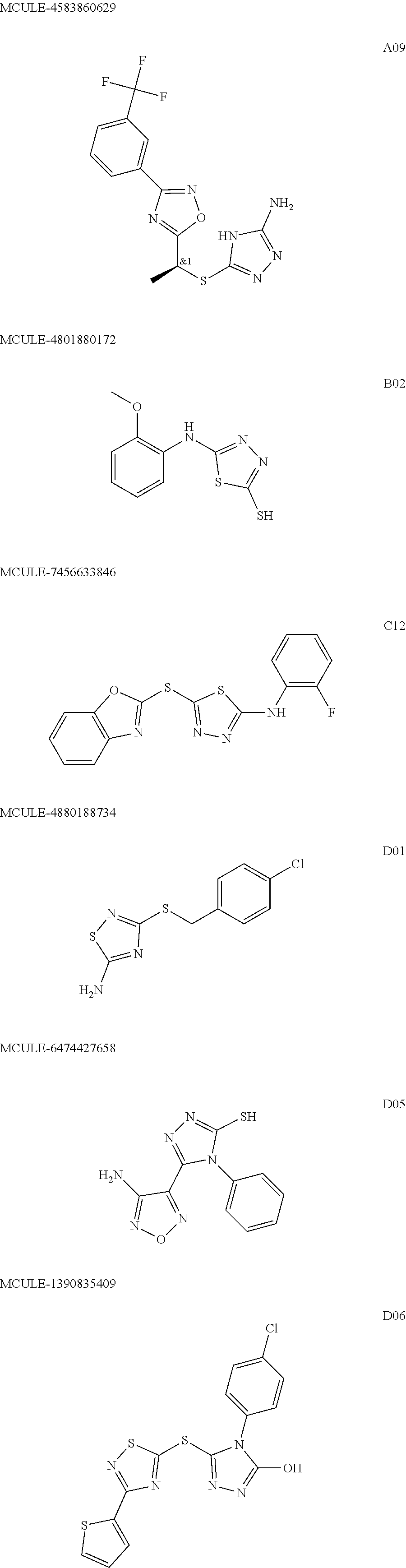 [SMILES: C[C@H](Sc1nnc(N)[nH]1)c1nc(-c2cccc(C(F)(F)F)c2)no1 ; COc1ccccc1Nc1nnc(S)s1 ; Fc1ccccc1Nc1nnc(Sc2nc3ccccc3o2)s1 ; Nc1nc(SCc2ccc(Cl)cc2)ns1 ; Nc1nonc1-c1nnc(S)n1-c1ccccc1 ; Oc1nnc(Sc2nc(-c3cccs3)ns2)n1-c1ccc(Cl)cc1]

| Matrix Plate | | | |
|---|---|---|---|
| Position | Mcule ID | SMILES | Chemical Names |
| A09 | MCULE- | CC(C1ON═C(C2═CC(C(F)(F)F)═CC═C2)N═1)SC1NC(N)═NN═1 | 5-((1-(3-(3- |
| | 4583860629 | | (trifluoromethyl)phenyl)-1,2,4- |
| | | | oxadiazol-5-yl)ethyl)thio)-4H- |
| | | | 1,2,4-triazol-3-amine |
| B02 | MCULE- | COC1═C(NC2SC(S)═NN═2)C═CC═C1 | 5-((2-methoxyphenyl)amino)- |
| | 4801880172 | | 1,3,4-thiadiazole-2-thiol |
| C12 | MCULE- | FC1═C(NC2SC(SC3OC4═C(C═CC═C4)N═3)═NN═2)C═CC═C1 | 5-(benzo[d]oxazol-2-ylthio)-N- |
| | 7456633846 | | (2-fluorophenyl)-1,3,4- |
| | | | thiadiazol-2-amine |
| D01 | MCULE- | N1═C(N)SN═C1SCC1═CC═C(C═C1)Cl | 3-((4-chlorobenzyl)thio)-1,2,4- |
| | 4880188734 | | thiadiazol-5-amine |
| D05 | MCULE- | N1ON═C(C═1N)C1N(C(S)═NN═1)C1C═CC═CC═1 | 5-(4-amino-1,2,5-oxadiazol-3- |
| | 6474427658 | | yl)-4-phenyl-4H-1,2,4-triazole-3- |
| | | | thiol |
| D06 | MCULE- | C1(═NN═C(O)N1C1═CC═C(Cl)C═C1)SC1SN═C(C2SC═CC═2)N═1 | 4-(4-chlorophenyl)-5-((3- |
| | 1390835409 | | (thiophen-2-yl)-1,2,4-thiadiazol- |
| | | | 5-yl)thio)-4H-1,2,4-triazol-3-ol |

A dosage unit for use of the selective inhibitor of 6-phosphofructo-2-kinase/fructose-2,6-biphosphatase 2 (PFKFB2) of the present disclosure, may be a single compound or mixtures thereof with other compounds. The compound may be mixed together, form ionic or even covalent bonds. The PFKFB2 inhibitor of the present disclosure may be administered in oral, intravenous (bolus or infusion), intraperitoneal, subcutaneous, or intramuscular form, all using dosage forms well known to those of ordinary skill in the pharmaceutical arts. Depending on the particular location or method of delivery, different dosage forms, e.g., tablets, capsules, pills, powders, granules, elixirs, tinctures, suspensions, syrups, and emulsions may be used to provide the PFKFB2 inhibitor of the present disclosure to a patient in need of therapy that includes the PFKFB2 inhibitor. The PFKFB2 inhibitor may also be administered as any one of known salt forms.

PFKFB2 inhibitor is typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the PFKFB2 inhibitor may be formulated to provide, e.g., maximum and/or consistent dosing for the particular form for oral, rectal, topical, intravenous injection or parenteral administration. While the PFKFB2 inhibitor may be administered alone, it will generally be provided in a stable salt form mixed with a pharmaceutically acceptable carrier. The carrier may be solid or liquid, depending on the type and/or location of administration selected.

Techniques and compositions for making useful dosage forms using the present disclosure are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 24th Ed., Lippincott Williams & Wilkins, 2000, and updates thereto; Martindale, The Extra Pharmacopoeia, Thirty-Second Edition (The Pharmaceutical Press, London, 1999); all of which are incorporated by reference, and the like, relevant portions incorporated herein by reference.

For example, the PFKFB2 inhibitor may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with an non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present disclosure include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the disclosure may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

The PFKFB2 inhibitor may be administered in the form of liposome delivery systems, e.g., small unilamellar vesicles, large unilamellar vesicles, and multilamellar vesicles, whether charged or uncharged. Liposomes may include one or more: phospholipids (e.g., cholesterol), stearylamine and/or phosphatidylcholines, mixtures thereof, and the like.

The PFKFB2 inhibitor may also be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylasparta-midephenol, or polyethyleneoxide-polylysine substituted with palmitoyl residues, mixtures thereof, and the like. Furthermore, the PFKFB2 inhibitor may be coupled one or more biodegradable polymers to achieve controlled release of the PFKFB2 inhibitor, biodegradable polymers for use with the present disclosure include: polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one aspect, gelatin capsules (gelcaps) may include the PFKFB2 inhibitor and powdered carriers, such as lactose, starch, cellulose derivatives, magnesium stearate, stearic acid, and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract.

For oral administration in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water-soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl- or propyl-paraben, and/or chlorobutanol. Suitable pharmaceutical carriers are described in Remington's Pharmaceutical Sciences, Mack Publishing Company, a standard reference text in this field, relevant portions incorporated herein by reference.

For direct delivery to the nasal passages, sinuses, mouth, throat, esophagus, trachea, lungs and alveoli, the PFKFB2 inhibitor may also be delivered as an intranasal form via use of a suitable intranasal vehicle. For dermal and transdermal delivery, the PFKFB2 inhibitor may be delivered using lotions, creams, oils, elixirs, serums, transdermal skin patches and the like, as are well known to those of ordinary skill in that art. Parenteral and intravenous forms may also include pharmaceutically acceptable salts and/or minerals and other materials to make them compatible with the type of injection or delivery system chosen, e.g., a buffered, isotonic solution. Examples of useful pharmaceutical dosage forms for administration of PFKFB2 inhibitor may include the following forms.

Capsules. Capsules may be prepared by filling standard two-piece hard gelatin capsules each with 10 to 500 milligrams of powdered active ingredient, 5 to 150 milligrams of lactose, 5 to 50 milligrams of cellulose and 6 milligrams magnesium stearate.

Soft Gelatin Capsules. A mixture of active ingredient is dissolved in a digestible oil such as soybean oil, cottonseed oil or olive oil. The active ingredient is prepared and injected by using a positive displacement pump into gelatin to form soft gelatin capsules containing, e.g., 100-500 milligrams of the active ingredient. The capsules are washed and dried.

Tablets. A large number of tablets are prepared by conventional procedures so that the dosage unit was 100-500 milligrams of active ingredient, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

To provide an effervescent tablet appropriate amounts of, e.g., monosodium citrate and sodium bicarbonate, are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Injectable solution. A parenteral composition suitable for administration by injection is prepared by stirring 1.5% by weight of active ingredient in deionized water and mixed with, e.g., up to 10% by volume propylene glycol and water. The solution is made isotonic with sodium chloride and sterilized using, e.g., ultrafiltration.

Suspension. An aqueous suspension is prepared for oral administration so that each 5 ml contain 100 mg of finely divided active ingredient, 200 mg of sodium carboxymethyl cellulose, 5 mg of sodium benzoate, 1.0 g of sorbitol solution, U.S.P., and 0.025 ml of vanillin.

For mini-tablets, the active ingredient is compressed into a hardness in the range 6 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

Kits. The present disclosure also includes pharmaceutical kits useful, for example, for the treatment of cancer, which comprise one or more containers containing a pharmaceutical composition comprising a therapeutically effective amount of PFKFB2 inhibitor. Such kits may further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the disclosure, unspecified materials and conditions are not excluded so long as they do not prevent the benefits of the disclosure from being realized.

When used as a chemotherapeutic agent the PFKFB2 inhibitor may also include, optionally, one or more potentiators that are typically mixed with a pharmaceutically acceptable carrier. This carrier can be a solid or liquid and the type is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents.

Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorings and coloring agents. Parenteral and intravenous forms may also include minerals and other materials to make them compatible with the type of injection or delivery system chosen.

As used herein, the term “chewable” refers to semi-soft, palatable and stable chewable treat without addition of water. It should be appreciated to the skilled artisan that a chewable composition will be stable and palatable, fast disintegrating, semi-soft medicated chewable tablets (treats) by extrusion without the addition of extraneous water. A soft chewable tablet does not harden on storage and are resistant to microbial contamination. A semi-soft chewable contain a blend of any one or more of binders, flavors, palatability enhancers, humectants, disintegrating agents, non-aqueous solvents, and diluents that are plasticized with liquid plasticizers, such as glycols and polyols to make them ductile and extrudable. The chewable can be made by extrusion, e.g., including fats or lipids as plasticizers and binding agents, is manufactured in the absence of added water, uses plasticizers to replace water in extrudable matrices, contains humectants to maintain the extrudable chew in a pliant and soft state during its shelf life, or any combination thereof. The chewable form may be provided in conjunction with one or more flavorings and/or taste masking agents that improve the taste of the formulation greater than 10, 20, 30, 40, 50, 60, 70, 80, or 90%. The chewable can include the active agent and the ion exchange resin to enhance taste masking.

For topical administration, the composition can be incorporated into creams, ointments, gels, transdermal patches and the like. The composition can also be incorporated into medical dressings, for example wound dressings e.g. woven (e.g. fabric) dressings or non-woven dressings (e.g. gels or dressings with a gel component). The use of alginate polymers in dressings is known, and such dressings, or indeed any dressings, may further incorporate the alginate oligomers of the disclosure.

As used herein, the term “cancer” refers to the ability to metastasize and metastasize a cell caused by uncontrolled cell division or to establish new growth at an extra site. The terms “malignant”, “malignant”, “neoplasm”, “tumor” and variations thereof refer to cancerous cells or groups of cancerous cells.

As used herein, the terms “anticancer agent”, “anticancer compound”, “anti-neoplastic compound”, “antitumor agent”, “anticancer therapy” and variations thereof are used interchangeably to refer to molecules that prevent cancer cells and tumor growth, including molecules that are capable killing cancer cells.

Specific types of cancer for targeting with the PFKFB2-specific inhibitors include, but are not limited to, glioblastoma multiforme, skin cancer, connective tissue cancer, fat cancer, breast cancer, lung cancer, stomach cancer, pancreatic cancer, ovarian cancer and genital cancer, cervix Cancer, uterine cancer, anogenital cancer, kidney cancer, bladder cancer, liver cancer, colorectal cancer or colon cancer and digestive (GI) duct cancer, prostate cancer and genital cancer, central nervous system (CNS) cancer, retinal cancer, blood cancer, lymphocyte cancer, and head and neck cancer.

Diseases or conditions of cardiac tissue, which expresses PFKFB2, can include myocardial ischemia, congestive heart failure, heart failure with preserved ejection fraction, or cardiac tissue regeneration.

Here the inventors show the recombinant expression, and unexpected serine phosphorylation of PFKFB2 in bacteria, which was subsequently used to optimize in vitro PFKFB2 kinase-specific activity. This assay was then used to screen an AI-generated library [2] of small molecules to identify a new inhibitor of PFKFB2 and PFKFB3.

Recombinant Protein Expression: PFKFB2, PFKFB1, and PFKFB3 constructs (Origene) were subcloned into GST expression vector. Mutation of PFKFB2 at K174 to G [3], H259 to A [4], and the double mutant were created using Quickchange methodology. All constructs were transformed into Rosetta cells (Milipore), expressed and induced following standard protocol. Bacteria were harvested via centrifugation and pellets resuspended in 10 mL sonication buffer (50 mM TrisHCl pH 7.5, 10% glycerol, 150 mM NaCl, 10 mM Imidazole, 10 uL 1M DTT, and 1 protease inhibitor tablet). Cells were sonicated (power lvl 5, 45 s, 4×, QSonica) at 4° C. and spun 10 krpm for 10 min at 4° C. to collect protein lysate. Glutathione-Sepharose beads (Cytiva) were washed twice with PBS and protein lysate was applied and rocked overnight at 4° C. Beads were captured using 10 mL column (Biorad) and washed 5× with column volume of PBS. Column was capped and incubated overnight with thrombin in 1 mL PBS at 4° C. 1 mL PBS was rinsed over the column 3× and protein was concentrated using Amicon Ultra 0.5 mL centrifugal filter (3K NMWL) (Millipore). Protein concentration was determined by Nanodrop (Thermo).

PKA and phosphatase treatments of recombinant protein: Bacterial constructs of PFK2 wild type and mutants were grown overnight in 1 ml Terrific Broth/Amp at 37 C with shaking. 500 ul of overnight culture was added to 10 ml Terrific Broth/Amp. Samples were grown 3 hrs at 37° C. with shaking. For induction, 100 μl of 0.04M IPTG was added to 10 ml TB/Amp. Samples were shaken overnight without heat. Cultures were pelleted and frozen at minus 80° C. Each pellet was resuspended in 1 ml sonication buffer. 50 mM Tris HCl pH 7.5, 10% glycerol, 150 mM NaCl, 10 mM imidazole, 10 μl 1M DTT, 1 protease inhibitor tablet). A 100 μl sample was taken from each resuspended pellet before sonication to represent 100% phosphorylation. The remaining sample was sonicated in 2×45 s bursts at level 4.5 using a tip sonicator. Assay conditions were 100 μl sonicated sample, 10 μl MnCl2 (10 mM NEB), 10 μl phosphatase buffer (10×PMP buffer, NEB) with or without 2 μl Lambda protein phosphatase (NEB, 400000 U/ml). Reactions were carried out for various times. To stop reactions, 30 μl of “stop” buffer mix was added (300 ul Invitrogen SDS 4× sample buffer, 10 μl 1M DTT, 5 μl Halt Protease and Phosphatase Inhibitor cocktail, Thermo Scientific). All stopped reactions were heated to 95° C. for 10 min. Samples were frozen at −80° C. for later western blot analysis.

Western blot analysis: Frozen samples were prepared for SDS PAGE by thawing and sonication (2×45 s). Samples were then diluted 1:4 in 1× sample buffer. 10 ul of each sample was run on SDS page (Invitrogen NP0321) and transferred to a nitrocellulose membrane, blocked for 1 hr with Odyssey TBS blocking buffer (LI-COR). Antibodies used were P-PFK2 Ser483 (rabbit mAB Cell Signaling) and P-PFKFB2 Ser466 (polyclonal Ab Invitrogen). Primary antibodies were incubated overnight at 4° C., secondary antibody (LI-COR IRDye800CW) was incubated 1 hr at room temperature. Blots were imaged on an Odyssey CLx System and analyzed using the Image Studio Software (LI-COR).

PFK-2 kinase activity assay: Relative in vitro PFK2 activities were assessed by the levels of fructose-2,6-bisphosphate (F-2,6-BP) produced by either 2.0 μg/mL of purified PFKFB2 (for plate-reader screening) or 5-20 ug/mL PFKFB2/PFKFB3 (for IC50 analyses) incubated in PBS on ambient temperature for one hour in the presence of 5 mM Mg²⁺, 1 mM ATP, and 1 mM fructose-6-phosphate (F-6-P). The resultant F-2,6-BP levels were determined by pyrophosphate-fructose-6-phosphate phosphotransferase (PPi-PFK) activity. After the preincubation phase, the F-2,6-BP/PFK2 mixture was diluted 10× by 50 mM pH 8.0 Tris assay buffer, supplemented with 5 mM Mg²⁺, 0.5 mM PPi and 1 mM F-6-P. The PPi-PFK activity was measured as the rate of NADH oxidation (ε₃₄₀=6200 M⁻¹ cm⁻¹) following the addition of 150 μM NADH to a mixture of excess PPi-PFK, 0.1 μg/mL triosephosphate isomerase, 10 μg/mL glycerol-3-phosphate dehydrogenase, and 0.01 U/mL aldolase. First 10 min of the reaction is an equilibration period and was discarded from rate calculations. PPi-PFK is the rate limiting step of this multi-enzyme reactions.

Generation of small molecule library: Small molecules were selected by the Atomwise ATOMNET® AI-platform trained upon the PFKFB2 crystal structure [5]. A total of sixty four (64) small molecules were selected and provided (blindly) as part of the Atomwise Aims program [2]. After the initial screen, molecules were unblinded and hits of interest were purchased for further validation and testing. Here, the inventors describe the identification of a PFKFB2 inhibitor referred to as B2 (5-[(2-methoxyphenyl)amino]-1,3,4-thiadiazole-2-thiol) purchased from Molport (Molport-000-869-332) for validation steps and cell culture treatments.

Cell culture and Seahorse XFe analysis: Human kidney carcinoma cell line A-498 (HTB-44) was obtained from ATCC. The effect of PFKFB2/3 inhibitors on glycolysis was determined by glycolysis stress test utilizing Seahorse XFe24 Extracellular Flux Analyzer (Agilent) measuring extracellular acidification rate (ECAR) at basal and maximal glycolysis. After cells reaching confluency in regular DMEM culture media, the media was replaced with either DMSO, 5 μM B2, 25 μM B2, or 5 μM PFK158-supplemented Seahorse media, which is comprised of basal unbuffered XF DMEM, 1 mM sodium pyruvate, and 2 mM glutamine (pH 7.4). During the assay, the following inhibitors were injected sequentially, as is standard for the glycolysis stress test: 10 mM glucose, 1 μM oligomycin, and 50 mM 2-deoxyglucose. After the Seahorse assay, without removing media, 1 μM of Hoechst 33342 was added to each well and subsequently the live cells were counted by Cytation 5 (Aligent) DAPI-channel fluorescent imaging for normalization purpose.

Targeted metabolomics of glycolytic intermediates: After A-498 cells reached 95% confluent in 75 cm² culture plates, culture media was replaced with DMEM containing either DMSO, 5 μM B2, or 5 μM PFK158. After 60 min incubation at 37° C. cells were once washed with warmed PBS and then quickly snap-frozen in LN2. Frozen cell culture plates, stored at −80° C., were taken out on dry ice/liquid nitrogen and 1.0 ml of chilled methanol (stored at −80° C.) was immediately added to the plate and plates were gently swirled for 1 minute on ice to make sure that all the cells were completely covered by methanol. Cells from the plates were then removed with a cell scraper and transferred to a 2.0 ml Eppendorf microcentrifuge tube. An additional 200 μl of chilled methanol was added to the plate and scraping was repeated for complete recovery of cells. Cells were then sonicated in a water bath for 5 min and then incubated for 20 min on dry ice and transferred to ice for thawing. 350 μl milli Q water was then added to the sample and shaken in a thermomixer for 10 min at 4° C. Cells were centrifuged at 15,000 rpm for 10 min and the supernatant was collected. Pellets were stored at −20° C. until use for protein estimation. The supernatant was filtered using Agilent Captiva EMR-lipid filter and filtrate was dried in a vacuum centrifuge for approximately 4 hours or until filtrate was completely dried. Dried samples were stored at −80° C. and then reconstituted in 100 μl of 7:3 v/v acetonitrile and water and transferred to LC/MS vials.

LC-MS analysis was performed on an Agilent 6546 LC/Q-TOF coupled to an Agilent 1290 Infinity II LC. Chromatographic separation was performed on an Agilent InfinityLab Poroshell 120 HILIC-Z, 2.1×150 mm, 2.7 μm column, coupled with a UHPLC Guard, HILIC-Z, 2.1 mm×5 mm, 2.7 μm, at 15° C., with a total run time of 29 min. The mobile phase consisted of 20 mM ammonium acetate buffer in water (pH=9.2) (A) and acetonitrile (B). To ensure a constant concentration during gradient elution, the InfinityLab deactivator additive (p/n 5191-4506) was added to the aqueous mobile phase. A linear gradient elution was used at a flow rate of 0.4 ml/min with the following program: 0.0 min (15% A and 85% B), 1.0 min (15% A and 85% B), 8 min (25% A and 75% B), 12.0 min (40% A and 60% B), 15.0 min (80% A and 20% B), 18.0 min (80% A and 20% B), 19.0 min (15% A and 85% B) and 29.0 min (15% A and 85% B). Data was acquired in a negative ESI full MS scan mode (scan range: m/z 40 to 1,000) using Agilent MassHunter Acquisition software version 10.0. Optimum values for MS parameters were as follows: gas temperature 300° C.; drying gas flow 13 L/min; Nebulizer pressure 40 psi; Sheath gas temperature 350° C.; Sheath gas flow 12 L/min; Capillary voltage, 3500V; Nozzle voltage OV; skimmer offset, 45V; Fragmentor 125V; Octopole 1 RF Voltage 750V.

Data analysis and peak integration for target metabolites were performed with Agilent MassHunter Quantitative analysis software (V10.1). An analytical standard mix of glycolytic and TCA cycle intermediates was analyzed along with samples for identification of the metabolites in the samples. For identification of target metabolites in the samples, a custom Agilent Personal Compound Database and Library (PCDL) of target metabolites (glycolytic and tricarboxylic acid cycle intermediates) was created from Agilent METLIN PCDL. The retention times derived from standard mix analysis were also included in the custom PCDL to facilitate the identification of metabolites in the samples. Relative abundance values for target metabolites were obtained by normalizing the raw data with total protein estimated from cell pellets obtained after methanol:water extraction using a BCA protein assay (Pierce).

Statistical Analysis: All statistical analyses were performed on GraphPad Prism Version 10.2.0. Principal component analysis was performed using PCAPlotter (scienceinside.shinyapps.io/PCAplotter/).

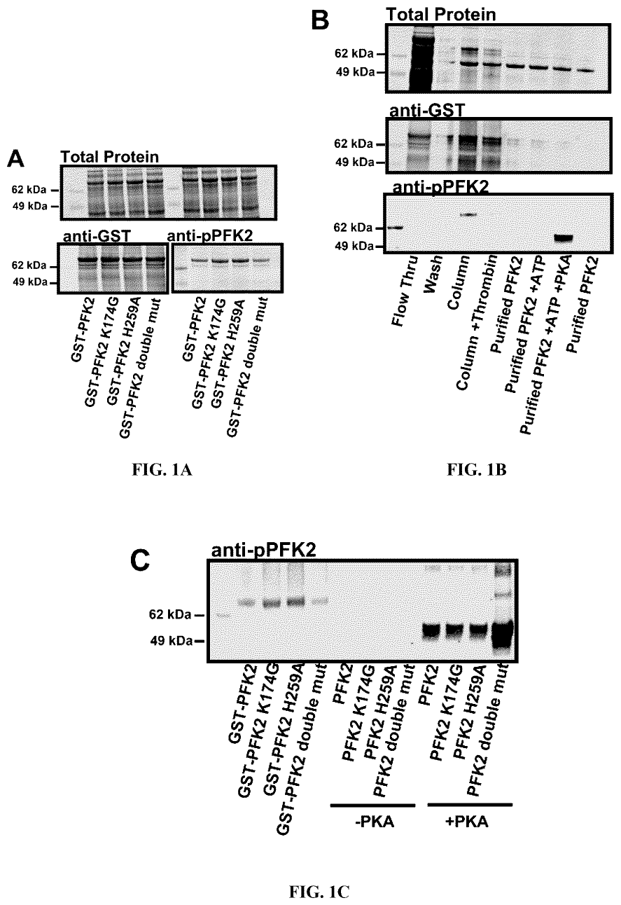

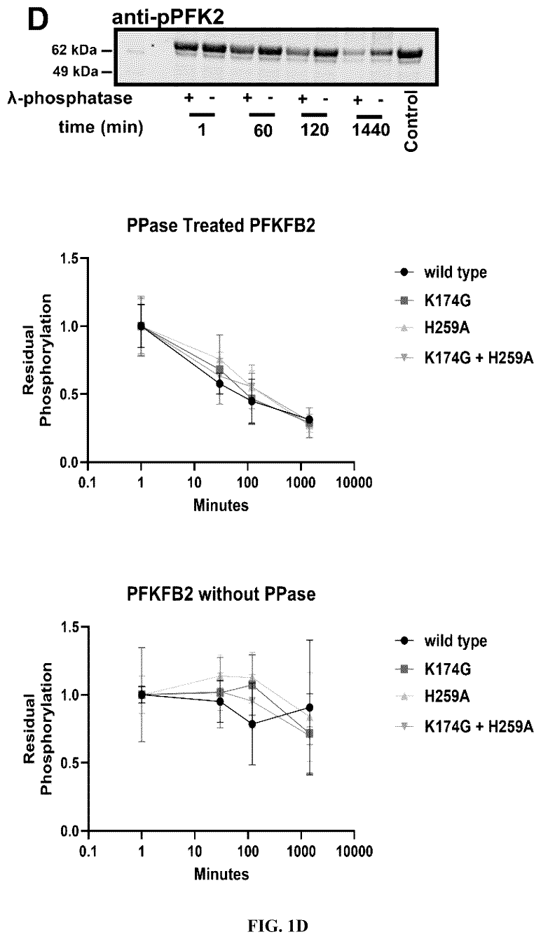

Bacterially expressed PFKFB2 is phosphorylated in situ: PFKFB2 was expressed to obtain enzymatically active protein that could be used for subsequent drug screens. PFKFB2 was bacterially expressed as a thrombin-cleavable GST fusion protein. While most of the PFKFB2 was insoluble, sufficient enzyme was enriched via a GSH-column and thrombin cleavage for subsequent experiments. FIG. 1A shows total cleared bacterial lysates probed with anti-GST antibody. FIG. 1B shows the enrichment of recombinant PFKFB2. In eukaryotes, PFKFB2 can be phosphorylated by several different serine/threonine kinases, including AKT, PKA, and AMPK [3]. This phosphorylation results in enhanced PFKFB2 kinase activity. Western blot analysis revealed that recombinant PFKFB2 purified from bacteria was phosphorylated at Ser483 (FIGS. 1A-1C). Phosphorylation was labile, though, with dephosphorylation occurring upon enrichment (FIG. 1B) and cleavage of PFKFB2 from the GST tag. However, purified PFKFB2 could then be rephosphorylated by treatment with exogenous PKA (FIGS. 1B and 1C) [6, 7].

The phosphorylation and dephosphorylation of PFKFB2 is not mediated via autonomous mechanisms: Bacteria are largely considered deficient in serine/threonine kinases, thus raising the possibility that PFKFB2 autophosphorylates. This also seems possible because there is an established precedence that metabolic small-molecule kinases can “moonlight” as protein kinases [8]. The inventors determined if intrinsic kinase activity of PFKFB2 was responsible for its in situ phosphorylation. Previous studies identified Lys174 within the ATP binding site as essential for kinase activity [3, 9]. Therefore, this critical residue to glycine (K174G; FIGS. 1A and 1C) was mutated. Interestingly, this mutation caused no change in the phosphorylation status of the protein upon purification (FIGS. 1A and 1C). In addition, the inventors created a phosphatase null PFKFB2 by mutating a critical histidine to alanine (H259A) in the phosphatase catalytic site [4], and a double mutant (K174G and H259A) lacking both kinase and phosphatase activities. In this manner, the inventors could also test whether dephosphorylation of PFKFB2 was also occurring in an autonomous manner. All three mutants expressed comparably and were phosphorylated in situ during bacterial expression (FIGS. 1A and 1C). Additionally, all constructs dephosphorylated during purification and could be rephosphorylated by PKA (FIG. 1C). These results support that PFKFB2 is phosphorylated in bacteria by an endogenous serine protein kinase activity [10].

Next, PFKFB2 (WT, K174G, H259A and double mutant) with a broad-specificity phosphatase (protein phosphatase lambda) were treated for increasing durations to further validate the phosphorylation status (FIG. 1D). These experiments were performed on crude bacterial lysates as this largely preserved the phosphorylation status of PFKFB2 recombinant protein. Western blot revealed PFKFB2 dephosphorylation occurred in a time-dependent manner in the presence of phosphatase (FIG. 1D). The rate and magnitude of dephosphorylation was similar between WT and the 3 mutants (FIG. 1D). In the absence of phosphatase, dephosphorylation was sporadic but occurred to a similar in magnitude between WT and the 3 mutants (FIG. 1D).

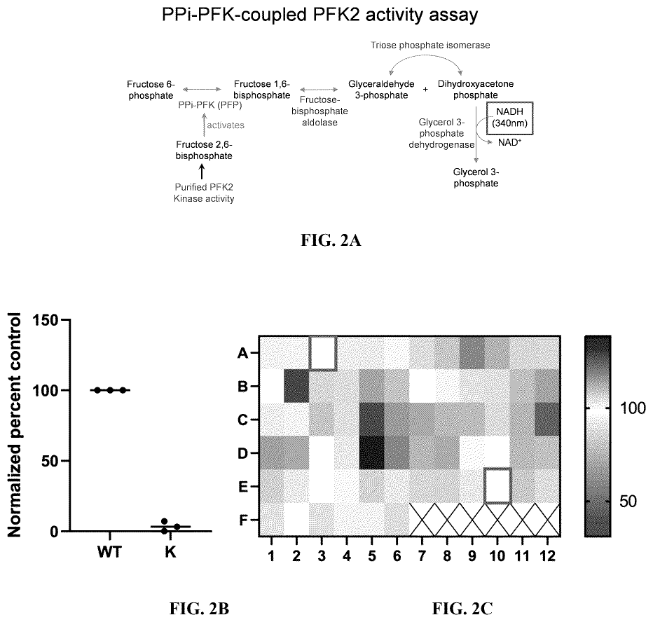

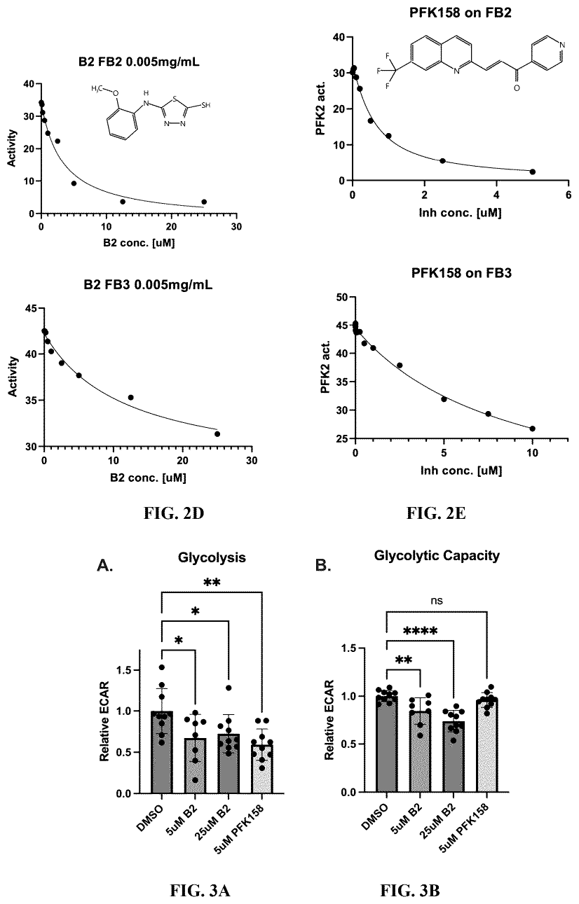

Identification of PFKFB2 modulators using a coupled kinase activity assay: Previous studies have used phsophofructokinase (PFK) isolated from potato tubers to measure fructose-2,6-bisphosphate (F-2,6-BP) levels [11]. The premise is that this plant isoform of PFK (PPi-PFK) requires the presence of F-2,6-BP for activity. Furthermore, PPi-PFK1 activity is proportional to the amount of F-2,6-BP present. The assay is ultimately coupled to glycerol-3-phosphate dehydrogenase (GPDH) activity and the oxidation of NADH, monitored spectrophotometrically at 340 nm (FIG. 2A). Here, this coupled assay was optimized to use as a readout of PFKFB2 kinase activity. As validation, mutant of PFKFB2 (K174G) that lacks kinase activity was compared to WT PFKFB2. As shown in FIG. 2B, K174G activity was negligible as compared to WT.

Next, the inventors sought to identify small molecule activators and/or inhibitors of PFKFB2. Putative molecules were selected based upon a modeling paradigm (Atomwise, San Francisco California [2]) that predicts interactions using a combination of AI and high-resolution protein crystallography data (obtained from [5]). 64 compounds were tested, each at 0.01 mM final concentration. A heat map, generated from the rates of WT PFKFB2 activity in the presence of testing compounds, demonstrates that several compounds were able to increase (blue) or inhibit (red) PFKFB2 activity as compared to DMSO control (FIG. 2C). The strongest activator (compound D5) was reobtained and investigated for further validation but failed to replicate the activation seen in the initial screen. The inventors further characterized the new inhibitor of PFKFB2 activity.

Validation of B2 as a new inhibitor of PFKFB2: The most potent inhibitor of PFKFB2 activity was in well B2 (FIG. 2C). This compound was unblinded as 5-[(2-methoxyphenyl)amino]-1,3,4-thiadiazole-2-thiol but referred to for simplicity as B2 in this work. See inset on FIG. 2D for molecular structure. A concentration course of B2 confirmed it inhibited PFKFB2 kinase activity with an IC50 of 3.289 μM (FIG. 2D). The inventors next tested whether B2 has specificity towards the PFKFB2 isoform or if it broadly inhibits PFKFB kinases. Recombinant PFKFB3 was therefore expressed and purified. A concentration course of B2 demonstrated it inhibited PFKFB3 with an IC50 of 11.89 μM (FIG. 2D). Other inhibitors of PFKFB kinase activity have been previously described but they have distinctly different chemical structures from B2. The inventors next sought to determine how B2 compares to one such established PFKFB inhibitor, PFK158 [12] a derivative of earlier inhibitor 3PO [13]. Under the same experimental enzyme assay conditions, a concentration course of PFK158 on recombinant PFKFB2 and PFKFB3 revealed IC50's of 0.629 μM and 8.939 μM, respectively (FIG. 2E). Thus, B2 is slightly less potent than PFK158 but these data validate B2 as an inhibitor of PFKFB2 and PFKFB3 kinase activities.

B2 decreases cellular glycolysis: The inventors next examined whether B2 can affect cellular glycolysis and metabolism. The inventors chose the A-498 (HTB44) cancer cell line because of its high rate of glycolysis and its expression of both PFKFB2 and PFKFB3 isoforms (confirmed by western blot)[14]. Seahorse XFe24 analysis of extracellular acidification rates (ECAR) revealed that both 5.0 and 25 μM B2 decreased basal glycolysis similarly. Furthermore, the magnitude of inhibition was similar to that induced by 5.0 μM PFK158 (FIG. 3A). However, there were differences between the inhibitors upon their effects on glycolytic capacity (FIG. 3B). Glycolytic capacity is the maximal rate of glycolysis achieved upon the addition of oligomycin. Both 5.0 and 25 μM B2, but not PFK158, inhibited glycolytic capacity significantly (FIG. 3B).

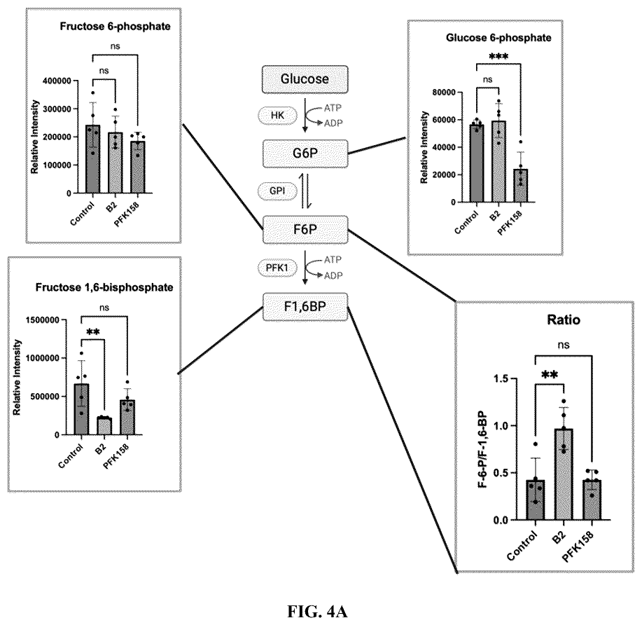

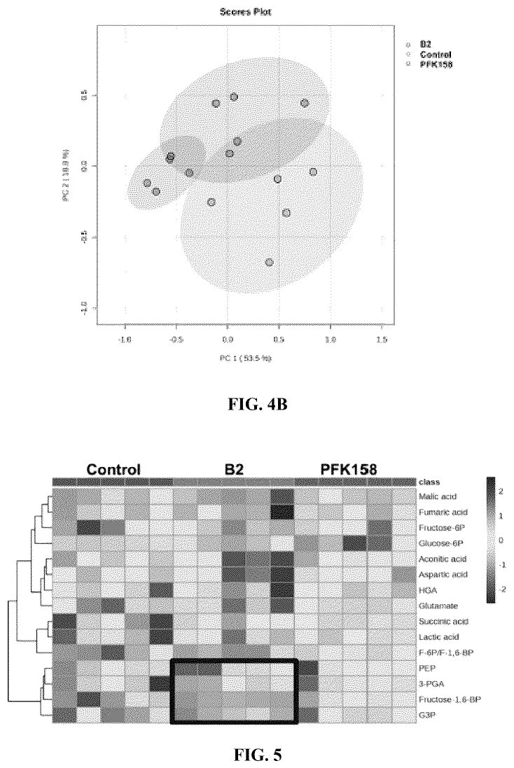

Next, the inventors performed LC-MS analysis on glycolytic intermediates extracted from A-498 cells that were treated with either 5.0 uM B2 or PK158 for 60 min. As shown in FIG. 4A, there were differential effects on early glycolytic intermediates. PK158, but not B2, caused a significant decrease in glucose-6-phosphate. Neither compound affected levels of fructose-6-phosphate. However, only B2 caused a significant decrease in fructose-1,6-bisphosphate and the ratio of fructose-6-phosphate to fructose-1,6-bisphosphate (FIG. 4A). This shows that B2 is inhibiting PFKFBs activation of glycolysis at the PFK1 step of glycolysis. The differential effects of B2 and PK158 on glycolytic intermediates are seen in principal component analysis of metabolites (FIG. 4B), demonstrating a clear distinction between B2-treated groups from control cells and cells treated with PK158. In conclusion, these results demonstrate that B2 is a glycolytic inhibitor with cellular metabolic bioactivity that is distinct from known PFKFB inhibitor, PK158.

The inventors sought to express recombinant PFKFB2 and develop a robust protocol to measure its kinase activity. Next, the inventors tested a library of small molecules, selected by a robust AI-platform, that target PFKFB2 kinase activity. The study resulted in the identification of new inhibitor that was further validated on both PFKFB2 and PFKFB3 kinase activities. Finally, the inventors showed that this newly identified PFKFB2 inhibitor decreases cellular glycolytic rates.

FIG. 5 shows a heatmap of targeted metabolomic data. Each column is a separate biological replicate (n=5). The box indicates the clustering of decreased downstream glycolytic intermediates in B2-treated cells. The ratio of fructose-6-phosphate (F-6-P) to fructose-1,6-bisphosphate (F-1,6-BP) is included in the heatmap. Other abbreviations: HGA, DL-hydroxyglutaric acid; PEP, phosphoenolpyruvic acid; 3-PGA, 3-phosphoglyceric acid; G3P; glyceraldehyde 3-phosphate; Data (n=5) are shown as mean±SD. Not significant (ns): **P≤0.01, ****P≤0.0001 by one-way ANOVA with multiple comparison of the mean of each test group to the mean of the vehicle control. PCA and heatmap were generated with MetaboAnalyst 6.0; created in BioRender.

An unanticipated result of the study was the finding that PFKFB2 expressed in bacteria is phosphorylated on Ser483. This is a well-characterized phosphorylation site that increases PFKFB2 kinase activity at the expense of decreased phosphatase activity. Bacteria are largely considered devoid of serine/threonine kinase activity, thus raising the intriguing possibility that PFKFB2 can autophosphorylate by “moonlighting” as a protein kinase. PFKFB4 can regulate transcriptional reprogramming by phosphorylating and activating the oncogenic steroid receptor coactivator-3 (SRC-3) protein [15]. However, it was found that this is not the case with PFKFB2. A kinase-null mutant was generated and purified from bacteria still emerged phosphorylated at Ser483. This shows that bacteria do indeed contain serine/threonine kinase activities. Furthermore, the dephosphorylation of PFKFB2 is labile and not likely due to intrinsic protein phosphatase activity. A phosphatase-null mutant of PFKFB2 still dephosphorylated in a time dependent manner upon purification.

The measurement of PFKFB2 kinase activity is challenging. The product, F-2,6-BP, is not stable and its in vivo concentration is low. Mass spectrometry detection of F-2,6-BP is further complicated by the lack of a commercially available standard. The current protocol for measuring F-2,6-BP is through a coupled assay that relies on the activation of PPi-PFK purified from potato tubers. The inventors have previously adapted this assay for measurement of F-2,6-BP levels in mouse hearts and here refined the assay to measure the activity of recombinant PFKFB2 kinase activity. Validation of the assay was achieved by showing that the kinase-null mutant of PFKFB2 lacked detectable activity. This allowed the inventors to screen small molecules that could potentially modulate PFKFB2 kinase activity.

The initial goal was to identify small molecules that could enhance PFKFB2 kinase activity because the inventors have previously shown that PFKFB2 expression and activation are decreased in the diabetic heart [16, 17] and increasing its kinase activity has protective effects against high fat diet-induced dysfunction [18, 19]. To do so, Atomwise used their proprietary AtomNet technology to target the bisphosphatase site, to determine whether inhibiting phosphatase activity may reciprocally enhance kinase activity of the bifunctional enzyme. For the virtual screen, the X-ray crystal structure of the human PFKFB2 dimer was used (PDB ID: 5HTK; [5]). PFKFB2 functions as a dimer where each bifunctional monomer is composed of four regions, with the core kinase and bisphosphatase domains at the center and 2 regulatory regions at the termini. The terminal regions regulate the enzyme's function by changing tertiary or quaternary conformations in response to several effectors [20] but are notably missing from the crystal structure. Sequence alignment of the human and mouse PFKFB2 shows a near conservation (˜90% identity) of residues forming the bisphosphatase domain (amino acids 249-505). Therefore, identified modulators have a high probability of targeting both human and mouse PFKFB2. The initial screen of small molecules showed one strong activator but failed further validation. Nevertheless, the inventors were able to identify several potential inhibitors of kinase activity. Given the interest and potential importance of PFKFB kinase inhibitors, this molecule was further characterized.

A potential limitation of a PFKFB kinase activity screen is that it is based upon a coupled assay. It is therefore possible that the small molecules being screened are interfering with one or more of the coupled enzymes (see FIG. 2A). To test this possibility, the inventors extracted F-2,6-BP from the hearts of Glyco^{Hi} mice. This is a transgenic mouse model that overexpresses a mutated form of PFKFB1 in the heart that has enhanced kinase activity and subsequently increased cardiac glycolysis [21]. The inventors have previously used the coupled assay employed herein to show that Glyco^{Hi} mouse hearts have persistently elevated F-2,6-BP levels [22]. In control experiments, it was found that B2 had negligible effects on F-2,6-BP measurements from Glycol^{Hi} mouse hearts (not shown). This demonstrates that B2 is directly affecting PFKFB kinase activity.

The PFKFB family of enzymes, and especially PFKFB3, are sought after targets for inhibition of kinase activity. This is because of their PFKFB3 inhibitors would specifically target cancer types that upregulate expression of PFKFB3 to increase glycolysis. The first and most widely used inhibitor is 3-(3-pyridinyl)-1-(4-pyridinyl)-2-propen-1-one (3PO) [13], while more recently other inhibitors based upon 3PO have been developed [23]. There are also applications outside of the heart as 3PO has shown to have therapeutic potential in a preclinical model of myocardial ischemia [24]. Interestingly, the molecule identified here (B2) is structurally distinct from 3PO. Indeed, it is demonstrated herein that B2 inhibits glycolysis in A-498 cells and causes a decrease in fructose-1,6-bisphosphate levels. This effect was distinct from the established PFKFB3 inhibitor PK158, a derivative of 3PO.

It is contemplated that any aspects of the disclosure discussed in this specification can be implemented with respect to any method, kit, reagent, or composition of the disclosure, and vice versa. Furthermore, compositions of the disclosure can be used to achieve methods of the disclosure.

It will be understood that particular aspects described herein are shown by way of illustration and not as limitations of the disclosure. The principal features of this disclosure can be employed in various aspects without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In aspects of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the disclosure(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any disclosure(s) in this disclosure. Neither is the “Summary” to be considered a characterization of the disclosure(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure but should not be constrained by the headings set forth herein.

All of the compositions and/or methods disclosed and claimed herein can be made and executed without undue experimentation in light of the present disclosure. While the compositions and methods of this disclosure have been described in terms of preferred aspects, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Yi, M., et al., 6-Phosphofructo-2-kinase/fructose-2,6-biphosphatase 3 and 4: A pair of valves for fine-tuning of glucose metabolism in human cancer. Mol Metab, 2019. 20: p. 1-13.
2. Atomwise, A. P., AI is a viable alternative to high throughput screening: a 318-target study. Sci Rep, 2024. 14(1): p. 7526.
3. Rider, M. H., et al., 6-phosphofructo-2-kinase/fructose-2,6-bisphosphatase: head-to-head with a bifunctional enzyme that controls glycolysis. Biochem J, 2004. 381(Pt 3): p. 561-79.
4. Argaud, D., et al., Adenovirus-mediated overexpression of liver 6-phosphofructo-2-kinase/fructose-2,6-bisphosphatase in gluconeogenic rat hepatoma cells. Paradoxical effect on Fru-2,6-P2 levels. J Biol Chem, 1995. 270(41): p. 24229-36.
5. Crochet, R. B., et al., Crystal structure of heart 6-phosphofructo-2-kinase/fructose-2,6-bisphosphatase (PFKFB2) and the inhibitory influence of citrate on substrate binding. Proteins, 2017. 85(1): p. 117-124.
6. Kitamura, K., et al., Phosphorylation of myocardial fructose-6-phosphate,2-kinase: fructose-2,6-bisphosphatase by cAMP-dependent protein kinase and protein kinase C. Activation by phosphorylation and amino acid sequences of the phosphorylation sites. J Biol Chem, 1988. 263(32): p. 16796-801.
7. Rider, M. H., et al., Evidence for new phosphorylation sites for protein kinase C and cyclic AMP-dependent protein kinase in bovine heart 6-phosphofructo-2-kinase/fructose-2,6-bisphosphatase. FEBS Lett, 1992. 310(2): p. 139-42.
8. Lu, Z. and T. Hunter, Metabolic Kinases Moonlighting as Protein Kinases. Trends Biochem Sci, 2018. 43(4): p. 301-310.
9. Bertrand, L., et al., Site-directed mutagenesis of Lys-174, Asp-179 and Asp-191 in the 2-kinase domain of 6-phosphofructo-2-kinase/fructose-2,6-bisphosphatase. Biochem J, 1997. 321 (Pt 3)(Pt 3): p. 623-7.
10. Pereira, S. F., L. Goss, and J. Dworkin, Eukaryote-like serine/threonine kinases and phosphatases in bacteria. Microbiol Mol Biol Rev, 2011. 75(1): p. 192-212.
11. Van Schaftingen, E., et al., A kinetic study of pyrophosphate: fructose-6-phosphate phosphotransferase from potato tubers. Application to a microassay of fructose 2,6-bisphosphate. Eur J Biochem, 1982. 129(1): p. 191-5.
12. Mondal, S., et al., Therapeutic targeting of PFKFB3 with a novel glycolytic inhibitor PFK158 promotes lipophagy and chemosensitivity in gynecologic cancers. Int J Cancer, 2019. 144(1): p. 178-189.
13. Clem, B., et al., Small-molecule inhibition of 6-phosphofructo-2-kinase activity suppresses glycolytic flux and tumor growth. Mol Cancer Ther, 2008. 7(1): p. 110-20.
14. Li, J., et al., Overexpression of PFKFB3 promotes cell glycolysis and proliferation in renal cell carcinoma. BMC Cancer, 2022. 22(1): p. 83.
15. Dasgupta, S., et al., Metabolic enzyme PFKFB4 activates transcriptional coactivator SRC-3 to drive breast cancer. Nature, 2018. 556(7700): p. 249-254.
16. Bockus, L. B. and K. M. Humphries, cAMP-dependent Protein Kinase (PKA) Signaling Is Impaired in the Diabetic Heart. J Biol Chem, 2015. 290(49): p. 29250-8.
17. Bockus, L. B., et al., Cardiac Insulin Signaling Regulates Glycolysis Through Phosphofructokinase 2 Content and Activity. J Am Heart Assoc, 2017. 6(12).
18. Newhardt, M. F., et al., Enhancing cardiac glycolysis causes an increase in PDK4 content in response to short-term high-fat diet. J Biol Chem, 2019. 294(45): p. 16831-16845.
19. Mendez Garcia, M. F., et al., Increased cardiac PFK-2 protects against high-fat diet-induced cardiomyopathy and mediates beneficial systemic metabolic effects. iScience, 2023. 26(7): p. 107131.
20. Okar, D. A., et al., PFK-2/FBPase-2: maker and breaker of the essential biofactor fructose-2,6-bisphosphate. Trends Biochem Sci, 2001. 26(1): p. 30-5.
21. Wang, Q., et al., Cardiac phosphatase-deficient 6-phosphofructo-2-kinase/fructose-2,6-bisphosphatase increases glycolysis, hypertrophy, and myocyte resistance to hypoxia. Am J Physiol Heart Circ Physiol, 2008. 294(6): p. H2889-97.
22. Batushansky, A., et al., GC-MS metabolic profiling reveals fructose-2,6-bisphosphate regulates branched chain amino acid metabolism in the heart during fasting. Metabolomics, 2019. 15(2): p. 18.
23. Jones, B. C., et al., Treatment against glucose-dependent cancers through metabolic PFKFB3 targeting of glycolytic flux. Cancer Metastasis Rev, 2022. 41(2): p. 447-458.
24. Yang, Q., et al., PFKFB3 Inhibitor 3PO Reduces Cardiac Remodeling after Myocardial Infarction by Regulating the TGF-beta1/SMAD2/3 Pathway. Biomolecules, 2023. 13(7).

What is claimed is:

1. A method of inhibiting 6-phosphofructo-2-kinase/fructose-2,6-biphosphatase 2 (PFKFB2) comprising:
contacting PFKFB2 with a molecule of formula: 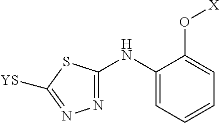
wherein X is CH₃, S, a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, a cyano group,
wherein Y is H, —SH, O, OH, O₂—R, S, a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, or a cyano group;
wherein R is a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, or a cyano group.

2. The method of claim 1, wherein the molecule has a reduced inhibitory effect against 6-phosphofructo-2-kinase/fructose-2,6-biphosphatase 3 (PFKFB3).

3. The method of claim 1, wherein the molecule is 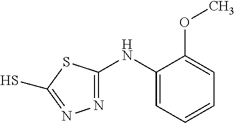
5-[(2-methoxyphenyl)amino]-1,3,4-thiadiazole-2-thiol, salts, and derivatives thereof.

4. The method of claim 1, wherein the molecule is formulated for intravenous, oral, enteral, parenteral, topical, intramuscular, subcutaneous, intradermal, transdermal, rectal, vaginal, mucosal, buccal, intraperitoneal, intranasal, sublingual, intranasal, intrapulmonary administration.

5. The method of claim 1, wherein the molecule further comprises one or more pharmaceutical acceptable carriers, excipients, buffers, binders, polymers, coatings, fillers, flavors, glidants, coatings, disintegrants, dyes, preservatives, sorbents, sweeteners, or salts.

6. The method of claim 1, wherein a dose for administration of the molecule is 0.01, 0.1, 1, 10, 20, 25, 30, 40, 50, 60, 70, 75, 80, 90, 100, 150, 150, 300, 333, 400, 500, 600, 700, 750, 800, 900, 1,000, 2,500, 5,000, 7,500, or 10,000 mg per dose.

7. The method of claim 1, wherein a dose for administration of the molecule is 0.01-0.1, 0.1-0.25, 0.1-0.4, 0.35-0.5, 0.5-1, 1-2, 1-3, 1-4, 1-5, 1-2.5, 2.5-3.5, 4-6, 5-8, 6-9, 7-10 grams per dose.

8. The method of claim 1, wherein the molecule is formulated for delivered orally via a mini-tablet, capsule, tablet, effervescent, dual release, mixed release, sachet, powder, or liquid.

9. The method of claim 1, wherein the molecule is formulated for daily doses of about 0.01, 0.1, 1, 2, 3, 4, 5, 10, 20, 25, 30, 40, 50, 60, 70, 75, 80, 90, 100 mg/Kg of body weight.

10. A method of treating a disease or condition with increased 6-phosphofructo-2-kinase/fructose-2,6-biphosphatase 2 (PFKFB2) activity comprising:
administering to a subject in need for the disease or condition an effective amount of a 6-phosphofructo-2-kinase/fructose-2,6-biphosphatase 2 (PFKFB2) inhibitor comprising of formula: 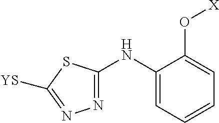
wherein X is CH₃, S, a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, a cyano group,
wherein Y is H, —SH, O, OH, O₂—R, S, a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, or a cyano group;
wherein R is a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, or a cyano group.

11. The method of claim 10, wherein the PFKFB2 inhibitor has a reduced activity against 6-phosphofructo-2-kinase/fructose-2,6-biphosphatase 3 (PFKFB3).

12. The method of claim 10, wherein the PFKFB2 inhibitor is: 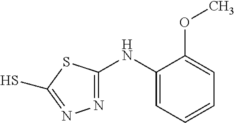
5-[(2-methoxyphenyl)amino]-1,3,4-thiadiazole-2-thiol, salts, and derivatives thereof.

13. The method of claim 10, wherein the PFKFB2 inhibitor is formulated for intravenous, oral, enteral, parenteral, topical, intramuscular, subcutaneous, intradermal, transdermal, rectal, vaginal, mucosal, buccal, intraperitoneal, intranasal, sublingual, intranasal, intrapulmonary administration.

14. The method of claim 10, wherein the PFKFB2 inhibitor further comprises one or more pharmaceutical acceptable carriers, excipients, buffers, binders, polymers, coatings, fillers, flavors, glidants, coatings, disintegrants, dyes, preservatives, sorbents, sweeteners, or salts.

15. The method of claim 10, wherein a dose of the PFKFB2 inhibitor for administration is 0.01, 0.1, 1, 10, 20, 25, 30, 40, 50, 60, 70, 75, 80, 90, 100, 150, 150, 300, 333, 400, 500, 600, 700, 750, 800, 900, 1,000, 2,500, 5,000, 7,500, or 10,000 mg per dose.

16. The method of claim 10, wherein a dose of the PFKFB2 inhibitor for administration is 0.01-0.1, 0.1-0.25, 0.1-0.4, 0.35-0.5, 0.5-1, 1-2, 1-3, 1-4, 1-5, 1-2.5, 2.5-3.5, 4-6, 5-8, 6-9, 7-10 grams per dose.

17. The method of claim 10, wherein the PFKFB2 inhibitor is delivered orally via a mini-tablet, capsule, tablet, effervescent, dual release, mixed release, sachet, powder, or liquid.

18. The method of claim 10, wherein the PFKFB2 inhibitor is administered in daily doses of about 0.01, 0.1, 1, 2, 3, 4, 5, 10, 20, 25, 30, 40, 50, 60, 70, 75, 80, 90, 100 mg/Kg of body weight.

19. The method of claim 10, wherein the PFKFB2 inhibitor is administered two or three times daily.

20. The method of claim 10, wherein the disease is a cancer selected from at least one of: glioblastoma multiforme, skin cancer, connective tissue cancer, fat cancer, breast cancer, lung cancer, stomach cancer, pancreatic cancer, ovarian cancer and genital cancer, cervix Cancer, uterine cancer, anogenital cancer, kidney cancer, bladder cancer, liver cancer, colorectal cancer or colon cancer and digestive (GI) duct cancer, prostate cancer and genital cancer, central nervous system (CNS) cancer, retinal cancer, blood Cancer, lymphocyte cancer, and head and neck cancer.

21. The method of claim 10, wherein the disease is myocardial ischemia, congestive heart failure, heart failure with preserved ejection fraction, or cardiac tissue regeneration.

22. The method of claim 10, wherein the PFKFB2 inhibitor decreased fructose-1,6-bisphosphate and increased fructose-6-phosphate levels in cells.

23. A molecule of formula: 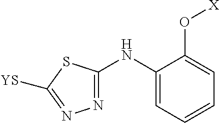
wherein X is CH₃, S, a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, a cyano group,
wherein Y is H, —SH, O, OH, O₂—R, S, a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, or a cyano group;
wherein R is a substituted or unsubstituted linear or branched alkyl group, a substituted or unsubstituted linear or branched alkoxy group, a substituted or unsubstituted linear or branched alkenyl group, a substituted or unsubstituted linear or branched alkenyloxy group, a substituted or unsubstituted linear or branched alkynyl group, a substituted or unsubstituted linear or branched alkynyloxy group, a substituted or unsubstituted cycloalkyl group, a substituted or unsubstituted cycloalkenyl group, an alkyl halide group, an alkyloxy halide group, or a cyano group.

24. The molecule of claim 1, wherein the formula is not: [chemical structure]
